(19) United States
(12) Patent Application Publication
(10) Pub. No.: US 2001/0037060 A1
(43) Pub. Date: Nov. 1, 2001

(54) Web site for glucose monitoring

(76) Inventors: Richard P. Thompson, Foster City, CA (US); Reid M. Rubsamen, Alamo, CA (US)

Correspondence Address: Karl Bozicevic, BOZICEVIC, FIELD & FRANCIS LLP, Suite 200, 200 Middlefield Road, Menlo Park, CA, 94025, US

(21) Appl. No.: 09/778,249

(22) Filed: Feb. 6, 2001

Related U.S. Application Data
(63) Continuation-in-part of application No. 09/500,377, filed on Feb. 8, 2000.

(51) Int. Cl. A61B 5/00; G06F 17/60

(52) U.S. Cl. 600/365; 705/3

(57) ABSTRACT

A computer implemented system for monitoring and analyzing data downloaded from a user's glucose monitor is disclosed. The computer system operates over the Internet to implement a data receptor form on screens accessible to a community of users. The system obtains manually entered data and receives the data downloaded from each user accessing the site presented to a community of users. Manually entered data such as times and components of meals and drug doses and times are plotted against the users glucose levels which are electronically downloaded into the system. Analyzed data are provided back to the user and/or the user's caregiver to provide information on how medications, foods, and exercise programs can be adjusted and timed to stabilize glucose levels and minimize symptoms from diabetes.

### CROSS-REFERENCE

This application is a continuation-in-part application of U.S. patent application Ser. No. 09/500,377, filed Feb. 8, 2000, which application is incorporated herein by reference.

### FIELD OF THE INVENTION

The invention relates generally to Internet systems and specifically to a computer generated interactive web site which allows a community of users to download data from their glucose monitors and have that data analyzed.

### BACKGROUND OF THE INVENTION

There are 15.7 million people or 5.9% of the U.S. population who have diabetes. Of those people about 10.3 million have been medically diagnosed with the disease while 5.4 million are unaware that they have the disease. Each day approximately 2,200 people are diagnosed with diabetes and about 798,000 people will be diagnosed with the disease this year. Based on death certificate data, diabetes contributed to more than 187,000 deaths in 1995.

For those with diabetes, there are a range of adverse effects caused by the disease. For example, diabetes is the leading cause of new cases of blindness in people ages 20-74 and causes 12-24 thousand people to lose their sight each year. Further, about 40% of all new cases of kidney disease is caused by diabetes and about 60-70% of those with diabetes have mild to severe forms of diabetic nerve damage. Each year over 56,000 amputations are performed on people with diabetes. Diabetics are 2-4 times more likely to have heart disease and to suffer a stroke as compared to non-diabetics.

There are a number of steps which can be taken to avoid the adverse effects of diabetes. These steps include regular exercise, proper diet and proper administration of medication. Although these steps will not cure diabetes, they can be effective in stabilizing glucose levels thereby minimizing adverse effects caused by the disease.

Regardless of the specific action taken to minimize adverse effects, the goal is to stabilize the patients' glucose levels over time. To assist in reaching that goal there are a number of glucose monitors available. Many of these monitors will not only allow a patient to accurately determine their glucose level but will electronically record measurements made and provide a means for downloading the recorded information. Although such monitors are available the electronic information within them has not been generally used. The present invention endeavors to facilitate the use of such information and provide a system which will assist users in managing the effects of diabetes.

### SUMMARY OF THE INVENTION

A computer generated web site is provided to a community of users over a communications system such as the Internet. The screen presented to users prompts the manual entry of data describing the user, the particular type of glucose monitor utilized and behavior of the user which would be expected to particularly affect the user's glucose level. The site also automatically recognizes the particular type of glucose monitor via information input by the user manually or electronically downloaded via a hardwire connection between the computer and glucose monitor. Upon recognition of the glucose monitor the site prompts the user to download data from the user's glucose monitoring device, which data are received by a program of the system designed to read the data from a number of different types of glucose monitoring devices. An analysis is carried out on both the manually entered data and that which is electronically downloaded from the glucose monitoring device. Results of the analysis may be provided back to the user in a manner which allows the user to readily appreciate the effects of exercise, meals, and medication and the effects each of these may have on various symptoms and on glucose levels. By entering information over time, the users can be provided with an analysis which will provide assistance to both users and their care givers on what behaviors are most beneficial toward maintaining desirable glucose levels.

An object of the invention is to provide a computer generated user interface system which receives and analyzes manually and electronically entered information relating to a user's glucose level.

A feature of the invention is that the system comprises a program which receives and interprets data from a number of different types of glucose monitors which are preferably automatically recognized by the site.

An advantage of the system is that manually entered data on the types, amounts and times of exercise, food, symptoms and medication can be analyzed with electronically downloaded data to provide users with results integrating one or both types of data which will provide assistance in stabilizing glucose levels over time.

An aspect of the invention is a user friendly web site interface allowing users such as diabetic patients to obtain an individualized analysis of behavioral and glucose level data to assist in stabilizing glucose levels over time.

Another aspect of the invention is that a screen interface generated for users can be accessed in different languages.

Yet another aspect of the invention is that the screen interface generated for the users can provide information on specific products and scientific information on specific topics of interest to the user.

Still another aspect of the invention is that the system preferably provides automatic recognition of a range of commercially available glucose monitors and generates a user screen preferably specific to the type of glucose monitor used.

These and other objects, advantages, and features of the invention will become apparent to those persons skilled in the art upon reading the details of the invention as more filly described below.

### BRIEF DESCRIPTION OF THE DRAWINGS

FIG. 1 is a flow chart depicting a browser-based model of the system of the present invention.

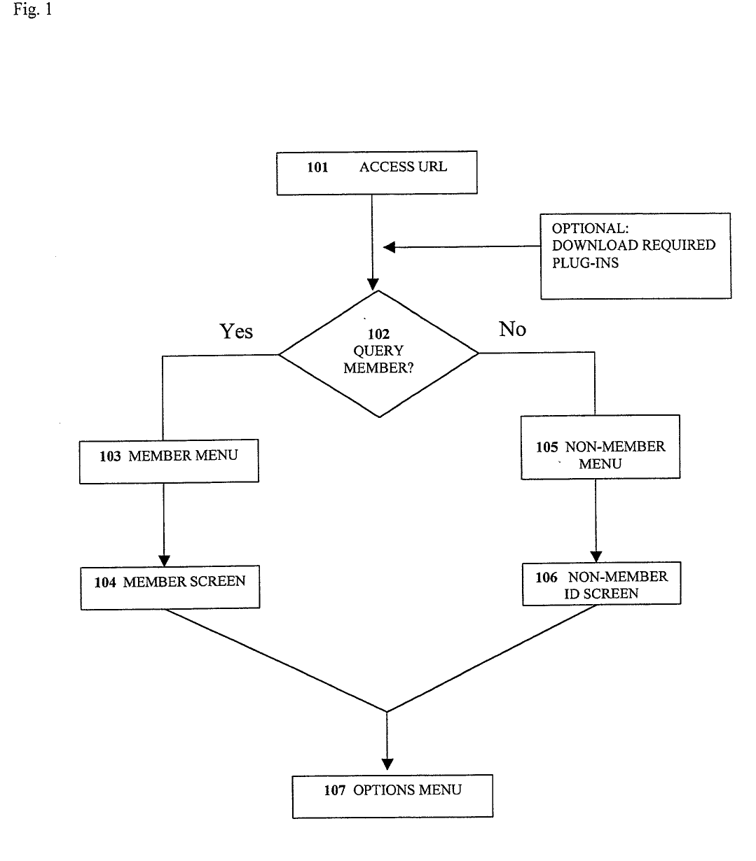

FIG. 2 is a flow chart depicting the user options for entry of information into the system of the present invention.

FIG. 3 is a flow chart depicting a software-based model of the system of the present invention.

### DETAILED DESCRIPTION OF PREFERRED EMBODIMENTS

Before the present systems are described, it is to be understood that this invention is not limited to particular systems or methodologies described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a User” includes a plurality of such Users and reference to “the measurement” includes reference to one or more measurements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “User” is used herein to mean any person. The user is preferably a person interested in obtaining information about glucose levels, diabetes, treatments relating to stabilizing glucose levels and/or determining the effects of various behaviors on glucose levels and symptoms associated with diabetes and/or impaired glucose homeostasis.

The term “diabetes” is used to describe a disease in which the body does not produce or properly use insulin, a hormone that is needed in the conversion of sugars, starches and other foods into energy needed in daily life. The term includes Type I diabetes, which is an autoimmune disease where the person does not produce any insulin. The term also includes Type II diabetes, which is a metabolic disorder resulting from the person's inability to make enough, or properly use, insulin. The term also includes gestational diabetes which occurs during some pregnancies and generally disappears when the woman is no longer pregnant. Further, the term includes other types of diabetes which result from genetic syndromes, surgery, injury, drugs, malnutrition, infections and other illnesses.

The terms “impaired glucose homeostasis” and “impaired glucose metabolism” refer to a condition in which blood glucose levels are above normal but not so high as to be classified as diabetes. For example, a person with a glucose level between 110 and 126 mg/dl after fasting for 8 hours could have impaired glucose homeostasis.

### General Overview of the Invention

Patients with diabetes have been able to self-measure their blood glucose using portable electronic monitors since the early 1980's. These devices accurately measure the glucose content of a blood sample applied to a sensor on the monitor. By performing frequent blood glucose measurements, a patient can better manage their disease by tailoring their diet and/or insulin therapy to their particular situation.

Although spot measurement of blood glucose provides valuable information to the patient, the long-term trend of blood glucose data over time, especially in relation to a history of when meals were taken, provides a more complete picture of the course of therapy. Most companies selling portable glucose monitors provide solid state memory within the monitor and a real-time clock to record the time and value of each blood measurement made. This stored data is typically made available directly on the monitor, via a scrolling function on the monitor's digital display or through a graphic trend showing multiple data points on the monitor's display.

In addition, many of the portable glucose monitors which contain memory provide a data port allowing the stored information to be transferred from the monitor to a personal computer. In this way, trend information can be displayed and/or archived, facilitating a more complete evaluation of blood glucose trend data than would be possible through the use of the limited storage and display capabilities afforded by typical portable glucose monitors.

Although the companies making portable glucose monitors with data download capability typically offer the cables and software necessary to transfer the monitor's memory to, for instance, a personal computer, this function is under utilized. Rarely do patients using portable glucose monitors with download capability actually ever download data stored within the monitor.

The reasons for this under-utilization are two fold. First, patients, although they may have the perception that such information in valuable, are not typically taught how to interpret blood glucose trend data and thus, may not know what to do with the data once it is available. Second, the technology for storing and tracking such data varies from monitor to monitor, and so it is not standardized for use by a medical healthcare provider. Because different patients may be using different brands of software for the data download function, a physician or the office staff would be confronted with having to match the patient's monitor with the appropriate brand of download software in order to routinely collect stored monitor data each time the patient returns for an office appointment. In addition, unless they are endocrinologists, many physicians, like the patients they treat, may not have any special training in blood glucose trend interpretation.

Another problem with current glucose monitor internal data storage systems is that they typically do not record ancillary information useful for clinical correlation with the blood glucose trend data. For example, the time meals were eaten and symptoms that might be associated with particularly high or particularly low blood glucose levels are not recorded with the insulin levels.

Another problem with current blood glucose monitor downloading hardware and software is that these were intended for local downloading of data. If, for example, a patient wished to transfer his or her blood glucose trend information to the treating physician, point-to-point electronic transmission of this data along, for example, telephone lines, would typically not be supported.

The present invention addressed the above-summarized concerns by providing a server-based system designed to communicate with client computers via the Internet. The server application is able to directly or indirectly query communication ports attached to the client computer so as to detect and identify the brand of a portable blood glucose monitor connected to the client computer. After the monitor identification is established, the server-based application will present an environment to the patient consistent with the brand of blood glucose monitor which they are using. In this way, familiar graphics, instructions, as well as other general information relevant to the patent's particular brand of blood glucose monitor is displayed. It is preferably that the user directly connect the glucose monitor to the user's computer using cables and software provided with the glucose monitor. The system provided here preferably includes a means for automatically recognizing the particular brand of monitor connected to the user's computer via electronically downloaded information. Once the particular glucose monitor is recognized the system generates a screen to the user which is particularly designed to correspond to that glucose monitor thereby making the system particularly user friendly.

Concurrently, the server will upload the contents of the blood glucose monitor's memory to the server site. Trend data will then be displayed in an appropriate format. The patient is asked to provide additional information not typically stored in the blood glucose monitor memory:

1. Overlay mealtime information (optionally including a description of what was eaten) onto the blood glucose trend data.

2. Overlay insulin-dosing information (time of day, dose and type of insulin taken).

3. Overlay symptom information (e.g. time of and severity of headache, polyuria, polydipsia, dizziness) onto the blood glucose trend data.

4. Overlay exercise information (e.g. time and type of exercise) into the blood glucose trend data.

To the extent that particular blood glucose monitors prompt for and store information regarding meal time data, insulin dosing and symptom information, these overlays could be constructed automatically using information stored in the blood glucose monitor's memory. In addition, to the extent that the means for administering insulin also contained electronic memory (electronic insulin “pen” injector, electronic insulin inhaler) this information could be downloaded separately to facilitate automatic completion of the insulin dosing information portion of the blood glucose trend overlay.

In addition, the server-based application will present the user with several options for the disposition of the data:

1. Store the trend data on the server site.

2. Email the trend data to the patient's physician for review.

3. Email the trend data to a physician associated with the site for review.

4. Subject the trend data to automatic interpretation.

5. Enter the trend data in a patient registry.

The information in the present system may be accessed in any number of ways, including but not limited to a browser based model and a download based model. Both of the aforementioned models involve a user accessing the system via URL, and entering information about their physical condition.

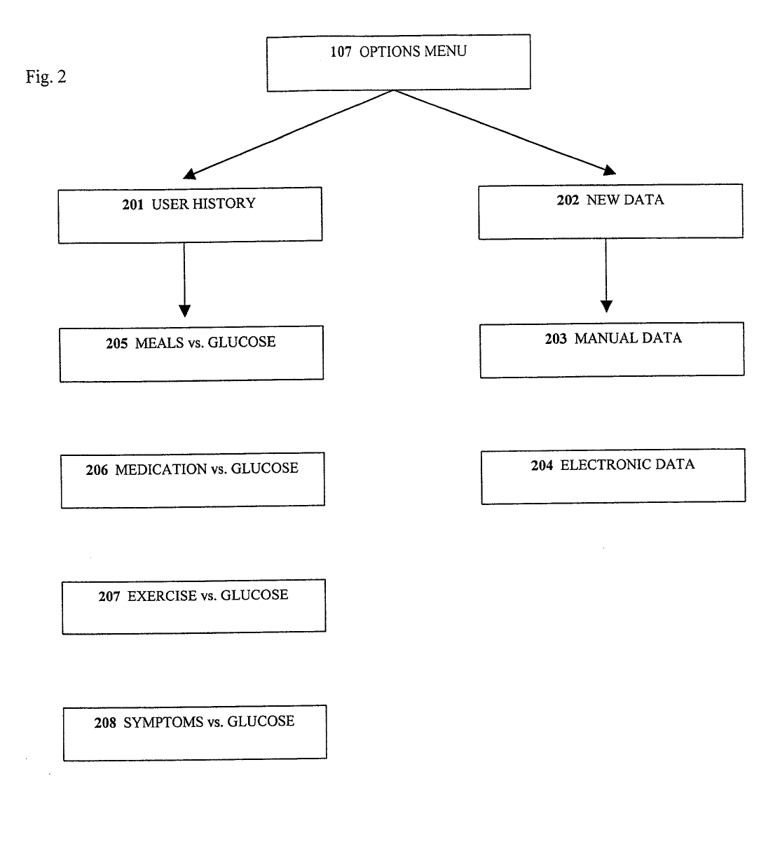

FIG. 2 shows an exemplary browser based system which begins with a user accessing the URL of the site 101. The user must access the system which is accessible to a community of users. The access is preferably over the world wide web of the Internet, although it may be accessible via other means such as subscribed user groups, bulletin boards, via specific Internet service providers, etc. The access address is entered to call up the web site on the user's screen. The access address is preferably a word or group of words or letter(s) which can be easily recalled and which more preferably relate in some manner to monitoring glucose levels, e.g., e-glucose.com or glucose.com or eglucose.com. Accessing the system may require a user to download plug-ins, such as QuickTime, and a user can optionally be prompted to download the required plug-ins prior to proceeding with the system. Accessing the system preferably includes receiving a signal from the user's computer which signal is specifically identified by the system as corresponding to a particular commercially available glucose monitor. Examples of commercially available glucose monitors include the following:

Glucose Monitors

| Company Name | Location | Name of Device |
|---|---|---|
| Roche Diagnotics/Boehringer | Switzerland | Accu-Chek |
| | | Advantage |
| Bayer Corporation | New York, CA | Glucometer ® |
| | | Dex ® |
| Amira Medical | Scotts Valley, CA | At Last ® |
| Lifescan | Miltpital, CA | One Touch ® |
| A Johnson & Johnson Company | | |
| Cygnus, Inc. | Redwood City, CA | Glucowatch ® |
| | | (soon to be |
| | | approved by |
| | | FDA) |
| Diagnostic Solutions, Inc. | Irvine, CA | Uni-Check ™ |
| | | Visual |
| TheraSense | Alameda, CA | FreeStyle ™ |

By automatically recognizing the type of glucose monitor used the system can be made more user friendly. Once the brand of glucose monitor is recognized the system will generate appropriately customized screens based on the brand.

Following acquisition of the necessary plug-ins (or if the necessary plug-ins are already downloaded), the system will query as to whether or not the user is a “member”, e.g., whether the user has entered and stored personal information previously, on a QUERY:MEMBER? screen 102. If the user is a member, the system then will display a MEMBER MENU 103 leading to a MEMBER SCREEN 104. The MEMBER SCREEN 104 requires the entry of information, e.g., a member identification number. Once the information is correctly entered it will send the user to an OPTIONS MENU 107 to choose from among various options for data entry which can be selected by the user.

The system as described, which requires user information be transmitted once to achieve a member identification number, is designed to minimize the possibility that confidential medical and personal information can be intercepted electronically. The invention described in U.S. Pat. No. 5,960,411 issued Sep. 28, 1999, is exemplary of this type of web site construction which minimizes transmissions of confidential information.

If the user is not a member, the system will display a NON-MEMBER MENU 105, leading to a non ID screen 106. This screen will require some identification from the user which may or may not be saved for future use (e.g., the non-member screen may allow a user to enter information to become a member). Once the non-member enters the required information, the user is then taken to the OPTIONS MENU screen 107.

The OPTIONS MENU 107 provides choices to the user relating to viewing an analysis of USER HISTORY 201 or entering NEW DATA 202 (See FIG. 2). The USER HISTORY 201 screen provides the user with several options for data analysis. For example, the electronically entered glucose level data can be plotted and overlaid or manually entered data on meals, medication, exercise, symptoms, and the like. The NEW DATA 202 screen provides the user with options regarding entering MANUAL DATA 203 and downloading ELECTRONIC DATA 204 directly from the user's glucose monitoring device. Once data has been entered, it is processed by a program of the central server which is designed to interpret data received from a number of different types of glucose monitoring devices.

A number of different parameters can be entered into the system, including but not limited to the user history and new data. User history can include direct comparisons such as MEALS v. GLUCOSE 205, MEDICATION v. GLUCOSE 206, EXERCISE v. GLUCOSE 207, and SYMPTOMS v. GLUCOSE 208. New data can include data entered manually by a user, e.g., keying in glucose levels measured by external means, or measurements may be directly electronically entered into the system, e.g., by hooking up a glucose monitoring device directly to an external port on the computer system and transferring the measurement into the system electronically.

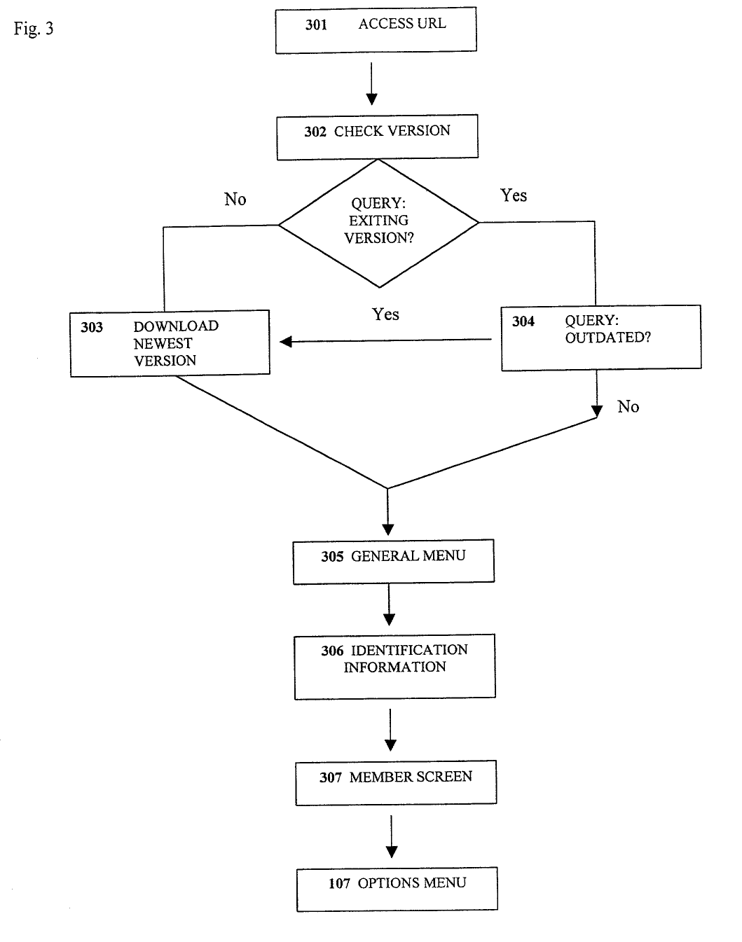

In one example of a download-based model (FIG. 3), a system is accessed by a user accessing a URL on an ACCESS screen 301. On the site will be access to the most recent software version of the system program for entering user information. The site will query the user as to the status of the software version downloaded by the user on a CHECK VERSION screen 302. If the user does not have any of the system software downloaded, the user will be directed to the most recent version of the glucose monitoring system program for downloading via a DOWNLOAD NEWEST VERSION screen 303. If the user does have a version of the system downloaded, the program will query as to the existing version (i.e. whether the user has the most updated version available) on a QUERY:OUTDATED? screen 304. If the user does not have the most updated version available, the user will be directed to the most recent version of the glucose monitoring system program for downloading. Once the most recent version has been downloaded, or if the user has already downloaded the most recent version of the program, then the user will be sent to a GENERAL MENU screen 305 for identification of the user and access to the system software. The menu options include, for example, choice of language, members or prior users, non-members or first users, general information about the web site, and general information of diabetes and/or specific products which might be used by diabetics.

If new software has been downloaded by a user, the user may be presented with a screen which requests basic identification and background health information from the user. The identification and background information is entered and stored for the downloaded system on an IDENTIFICATION INFORMATION screen 306. The system then assigns the user an identification code which can be entered in a MEMBER SCREEN 307 thereby eliminating the need to send confidential information over the Internet more than once. If the user is not accessing the software for the first time, the software may skip the IDENTIFICATION INFORMATION screen 306 and go directly to the MEMBER SCREEN 307.

Once the required information is entered, the user is sent to an OPTIONS MENU screen 107 such as that described in reference to FIG. 2. This OPTIONS MENU screen 107 allows the user to enter information regarding health and glucose levels as in the browser-based system. The information entered by the user can be transmitted back to the original system accession site for computation and analysis, storage, accession by a third-party (e.g., a medical care provider), etc. Alternatively, the information can be stored on a user's personal computer, either directly or following analysis using the system.

The server-based system allows one or more remote computers to access simultaneously a local computer network, even if each of the remote computers employs a different protocol (e.g., IPX, TCP/IP, AppleTalk, NetBEUI, or 802.2/LLC). Theoretically, use of the JAVA programming language allows for smooth and versatile communication between computing systems even when the computing systems are operating on different hardware platforms and are using different operating systems, so use of this languager is preferred. In addition, software for translation of the contents into various languages may be employed, such as Uni-Verse™ or InterTran™.

In one embodiment, each user is provided with a card, preferably a “smart card,” which has a memory and, preferably, an onboard processor. The card is encoded with the respective user's identifying information, and preferably includes other data such as card access authorization codes, information that identifies at least one remote network address of at least one remote facility where data records are stored, and specific user file locations at each network address. User data, such as health or financial data for any number of patients or members, is stored in a data base at one or more remote facilities, for example, at one or more hospitals or one or more central processing facilities.

### Glucose Monitors

There are a number of different types of glucose monitors sold commercially and others are being developed. The system of the present invention is designed to be interactive with a range of different types of glucose monitors. Glucose monitors and related materials are known to those skilled in the art as described and disclosed in U.S. Pat. Nos. 5,463,467; 5,334,508; 5,382,523; 4,477,575; 4,816,224; 5,424,035; 5,206,147; 5,240,860; 4,731,726; 5,942,102; 5,843,691; 5,753,452; 5,563,042; 5,972,294; 5,789,255 as well as the patents and publications cited in the above listed patents.

In addition to providing a means to download and analyze data from one of different types of glucose monitoring devices, the system of the invention can provide the user with a direct means of monitoring glucose levels and/or testing for whether the user is at risk for diabetes and/or has a symptom of diabetes. This includes tests for conditions that can be surrogate measurements for diabetes. For example, tests for glaucoma can be conducted using a computer screen as taught in U.S. Pat. No. 5,061,059 issued Oct. 29, 1991 and U.S. Pat. No. 5,946,075 issued Aug. 31, 1999. The invention allows for the generation of an image available on a screen or monitor which can be used to detect the presence of eye disease in the human eye. Users presented with a fixation target positioned on a colored planar surface. The user focuses a test eye on the fixation target and positions the test eye at sufficient separation distance between the fixation target and aligns the test eye relative to the fixation target so that the USER's central and peripheral vision health can be tested. Additional marks are presented on the planar surface of the screen for detection by the user using the peripheral vision of the test eye. The additional marks are primarily the same level of black-white contrast as the planar surface, but different in hue to create color contrast symbols, and are presented within the field of vision of an eye not afflicted with the disease for which testing is being conducted. In this way, the presence of the eye disease can be detected if the additional marks are not visible to the test subject. In order to best utilize such a system, it is preferable to have a large screen (e.g., a screen which is approximately 17 inches or more in size).

Further, a user's glucose level could be tested directly over the system of the present invention by using technology of the general type taught in U.S. Pat. No. 5,713,353 issued Feb. 3, 1998. The system for testing blood glucose levels over the Internet can be carried out by projecting onto the screen a changing light pattern which changes in regard to one or several parameters defining its luminescence, color, rate of flicker, spatial contrast, detailed content, speed or otherwise provided that the change gradually stimulates one retinal system more than another retinal system. The user observes the changing light pattern until a subjective visual effect occurs, which is preferably the user's perceived dramatic change in the appearance of the light pattern, for example a change in the perceived direction of rotation. The subjective visual effect indicates a specific ratio of stimulation of the two retinal systems, for example the point of balance of stimulation of the two systems. Upon noting the subjective visual effect, the user activates a signal sent back to the system (e.g., clicks the mouse). This signal takes note of the current light pattern parameter values, allowing the system to relate such parameter values with a corresponding blood glucose level using predetermined calibration data. Accordingly, the user's glucose level is accurately determined in a completely non-invasive manner. This glucose level measurement can be entered into the patient's history and compared with glucose levels taken by a more conventional blood glucose monitoring system in order to calibrate the non-invasive system.

### Remote Access

The present invention can also be used to allow a third party (e.g., a physician, health care provider, nutritionist, etc.) to access a user's information remotely. Remote access is provided from a remote host system to an instrument. A remote user generally can utilize any type of computer to access the network. The computer can be, for example, a personal computer, a workstation, or a portable computer such as a laptop computer or a notebook computer. Also, the computer can be, for example, an IBM PC or compatible, an Apple Macintosh, or a Unix-based computer. The user typically connects a modem or similar communication device to a serial port of the computer. The modem connected to the user's remote computer communicates over the telephone lines with another modem which is coupled to the server or database containing a user's integrated information. The other modem and the server are located at the network which the remote computer is attempting to access. The server is coupled directly to the network. It is the server which provides the remote computer with controlled access to the network and the services and resources thereon. The server is referred to as a “remote access server,” and it typically includes a serial port for connecting to the other modem, a port for connecting to the network, and electronics which include at least a microprocessor and memory.

For example, if the control and data acquisition software is run on the remote host system, a user of the remote host system is provided with a graphical user interface which the user can utilize to interact with the instrument. The data received from the instrument by the remote host system is displayed on the host system. The data may be additionally processed at the remote host system before being displayed. See e.g., U.S. Pat. No. 5,790,977. The control and data acquisition software can run as an application within an internet browser. In this case, the control and data acquisition software can be forwarded to the remote host system in response to a HyperText Transfer Protocol (HTTP) server command. Preferably, a user's software can run on any internet browser on almost all platforms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. In a computer system which implements a data receptor form on a screen which is accessible to a community of users, a method of analyzing data on glucose levels, the method comprising the computer implemented steps of:
(a) providing user access to a community of users;
(b) generating a screen for a user access wherein the screen prompts the user to manually enter data;
(c) obtaining data manually entered as prompted;
(d) receiving data from a glucose monitoring device of the user wherein a system which receives the data from the glucose monitor comprises a program which recognizes data downloaded from a plurality of different types of glucose monitoring devices;
(e) analyzing the data from the monitoring device and data manually entered; and
(f) producing a result based on the analysis.

2. The method of claim 1, wherein the user access is provided by means of world wide web access.

3. The method of claim 2, wherein the system is a browser-based system.

4. The method of claim 2, wherein the system is a software-based system.

5. The method of claim 1, wherein the manually entered data comprises data selected from the group consisting of: user name, age, sex, weight, height, race, current medications, medication dosages, time of meals, food eaten at meals, symptoms, and caregiver name.

6. The method of claim 1, further comprising:
sending the result of the analysis to the user.

7. The method of claim 1, further comprising:
sending the result of the analysis to a caregiver.

8. The method of claim 1, further comprising:
repeating (a), (b), (c), (d) and (e) a plurality of times over a period of days; and
storing data entered manually and data received from the user's glucose monitoring device.

9. The method of claim 8, further comprising:
plotting data received from the user over a period of days thereby creating a graph from the data.

10. The method of claim 9, further comprising:
comparing data manually entered against the graph in a manner which allows for a visual representation of events affecting the user's glucose level.

11. The method of claim 10, wherein the manually entered data compared against the graph are data relating to meals eaten by the user.

12. The method of claim 10, wherein the manually entered data compared against the graph are data relating to medication administered by the user.

13. The method of claim 10, wherein the manually entered data compared against the graph are data relating to user symptoms.

14. A method of analyzing data from a glucose monitor, comprising the steps of:
connecting a hand-held glucose monitoring device to a computer;
connecting the computer via the internet to a remote web site;
analyzing data at the remote web site which data is sent from the glucose monitoring device to the computer and then to the remote web site;
determining characteristics about the glucose monitoring device based on the data analyzed.

15. The method of claim 14, further comprising:
sending data from the web site to the computer which data generates an image on a screen connected to the computer which image is specific to a characteristic of the glucose monitoring device.

16. The method of claim 15 wherein the characteristic of the glucose monitoring device is specific to a commercial brand of glucose monitoring device.

17. The method of claim 15, further comprising:
electronically downloading information from the glucose monitoring device to the computer;
sending the information from the computer to the web site.

18. The method of claim 17, further comprising:
analyzing the information at the web site; and
sending signals to the computer in order to generate an informational image on the screen based on information analyzed.
